(19) United States
(12) Patent Application Publication
(10) Pub. No.: US 2010/0023118 A1
(43) Pub. Date: Jan. 28, 2010

(54) METHOD AND APPARATUS FOR REPAIRING OR REPLACING CHORDAE TENDINAE

(75) Inventors: Marilyn Medlock, Irvine, CA (US); Dan Howk, Irvine, CA (US); Greg Bak-Boychuk, San Clemente, CA (US); Chris Okos, Huntington Beach, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/179,385

(22) Filed: Jul. 24, 2008

(51) Int. Cl. A61F 2/24; A61B 19/00; A61B 17/04

(52) U.S. Cl. 623/2.11; 128/898; 606/228; 606/232

(57) ABSTRACT

A method and apparatus for performing mitral valve chordal repair on a patient include attaching at least one filament to a mitral valve leaflet and to a papillary muscle. The length of filaments can be adjusted by adjusting tension in a filament or by altering the effective length of a filament by cutting filament strands or by moving an adjustment member along the length of the filaments.

### BACKGROUND

The present disclosure concerns methods and apparatuses for replacing, repairing, or supplementing chordae tendinae to improve or restore the connection of the mitral leaflets to the heart wall.

### DESCRIPTION OF THE RELATED ART

Mitral regurgitation is a valvular heart disease that results in the abnormal leaking of blood through the mitral valve, from the left ventricle into the left atrium of the heart. The mitral valve includes valve leaflets and a mitral valve annulus that forms a ring around the valve leaflets. Chordae tendineae connect the valve leaflets to the papillary muscles, which tether the valve leaflets to the left ventricle and prevent them from prolapsing into the left atrium. Mitral regurgitation can result from the dysfunction of any of these portions of the mitral valve.

In some patients with mitral regurgitation, the abnormal leakage of blood is caused, at least in part, from damaged chordae. The chordae can be, for example, elongated or torn, which can cause the mitral valve to function improperly. Artificial chordae can be used to supplement or replace damaged chords to attempt to improve mitral valve functioning. It is important that the artificial chordae be selected to be a proper length so that they serve the desired purpose and, at the same time, do not cause additional stresses to the heart itself. Known methods of implementing artificial chordae, however, fail to provide precise mechanisms for adjusting the length of the artificial chords while maintaining the anatomy of both the valve and the papillary muscle.

Moreover, traditional methods of chordae replacement often require patients to undergo open heart surgery with a bypass machine. In addition to being highly invasive and causing significant stress and trauma to the patient, these methods require surgeons to estimate proper chordae length in an environment that does not properly reflect the normal beating heart.

### SUMMARY

The present disclosure is directed toward new and non-obvious methods and apparatuses for performing mitral valve chordal repair on a patient while the patient's heart is beating.

In one embodiment, a method is disclosed that includes inserting a catheter that contains at least a first and second filament into a left ventricle of a patient. A first end of the first filament is attached to a mitral valve leaflet at a valve attachment site, and a first end of the second filament is attached to the mitral valve leaflet at or near the same location as the attachment of the first end of the first filament. The second end of the first filament is attached to a first attachment site at either a papillary muscle or along the ventricular wall, and a second end of the second filament is attached to a second attachment site at either a papillary muscle or the ventricular wall. The first and second attachment sites are different from one another. An adjustment device is attached to both the first and second filaments, and the adjustment device is configured to hold portions of the first and second filaments in close proximity to each other at the area where the adjustment device contacts the first and second filaments. The adjustment device is movable along a length of the first and second filaments to adjust the tension in the filaments.

The adjustment device can be moved along a length of the first and second filaments such that the movement of the adjustment device causes the effective length of both the first and second filament to be either lengthened or shortened. The adjustment device can be locked at a desired location along the first and second filaments, in which state it holds the first and second filaments securely together. The adjustment device optionally can be a clamp with interlocking teeth that are configured to lock the clamp in one or more positions of varying circumference.

Optionally, the adjustment device can be a tension adjustment block. The tension adjustment block can comprise a pin member and ring member. The pin member and ring member can be configured to lock together in a locked state when the pin member is pushed into the ring member. In addition, the pin member and the ring member optionally can be unlocked from the locked state so that the act of moving the adjustment member can be performed more than once.

The pin member and ring member optionally can be unlocked by inserting an adjustment catheter between at least a portion of the pin member and at least a portion of the ring member, and applying a separating force to one or both of the pin member and the ring member to separate the pin member from the ring member. In addition, the adjustment catheter optionally can comprise a fork member having one or more prongs, an inner push member, and an outer push member. The one or more prongs can comprise the portion of the adjustment catheter that is inserted between a portion of the pin member and a portion of the ring member. The inner push member can be contained in an area of the adjustment catheter, and the outer push member can be of a greater diameter than the inner push member so that it surrounds a portion of the inner push member. The inner push member can be configured to engage the adjustment member to unlock the pin member from the ring member, and the outer push member can be configured to engage the adjustment member to lock the pin member to the ring member.

In another embodiment, a method is disclosed that includes inserting a catheter into a left ventricle of a patient. The catheter contains at least one suture and the suture comprises at least two filament strands. The filament strands are attached at a first common area at one end of the filament strands and at a second common area at the other end of the filament strands. The respective lengths of the filament strands between the first and second common areas are different from one another. A first end of the suture is attached to a mitral valve leaflet and a second end of the suture is attached to a papillary muscle or a ventricular wall. The effective length of the suture is adjusted by cutting one or more of the filament strands.

Optionally, the suture can comprise four or more filament strands. The difference between the length of the shortest filament strand and the longest filament strand optionally can be greater than 13 mm.

In another embodiment an apparatus is disclosed for securing two or more artificial chordae filaments together such that an effective length of the filaments can be adjusted. The apparatus comprises a pin member and a ring member. The pin member and ring member have openings for receiving at least two filaments. The ring member can be configured to receive at least a portion of the pin member. The apparatus can be placed in a locked state by inserting the pin member into the ring member, thereby capturing the filaments between the pin member and the ring member such that the pin member and the ring member are held at a fixed position relative to the filaments. The apparatus can be placed in an unlocked state by separating the pin member and the ring member so the pin member and the ring member can be moved relative to the filaments.

In another embodiment a system for adjusting an effective length of artificial chordae within a patient is disclosed. The system comprises an adjustment member and a tool configured to lock and unlock the adjustment member. The adjustment member is configured to secure two or more artificial chordae together. The adjustment member comprises a pin member and ring member. The pin member has a first end and a second end, and an opening for receiving at least two filaments. The ring member has an opening for receiving the at least two filaments and is configured to receive at least a portion of the first end of the pin member. The tool comprises a fork member positioned at the distal end of the tool with one or more prongs, an inner push member, and an outer push member. The inner push member is contained within a lumen of the tool. The outer push member is of a greater diameter than the inner push member and surrounds at least a portion of the inner push member. The tool is configured to manipulate the adjustment member between a locked and an unlocked position.

In another embodiment, an apparatus is disclosed for securing a mitral valve leaflet to a papillary muscle. The apparatus comprises at least two filament strands that are attached at a first common area at one end of the filament strands and at a second common area at the other end of the filament strands. The respective lengths of the filament strands between the first and second common areas are different from one another.

The apparatus optionally can include four or more filament strands. The difference between the length of the shortest filament strand and the longest filament strand optionally can be 13 mm or greater. The filament strands optionally can contain different markings that are visible under fluoroscopy so that the filament strands can be distinguished from one another via fluoroscopy.

The foregoing and other features and advantages will become more apparent from the following detailed description, which proceeds with reference to the accompanying figures.

### BRIEF DESCRIPTION OF THE DRAWINGS

FIG. 1 is a schematic view of another embodiment of a method and apparatus for attaching artificial chordae to valve leaflets.

FIG. 2 is an illustration showing an adjustment of the effective length of artificial chordae according to one embodiment.

FIG. 3 is a view of an embodiment showing an adjustment member.

FIG. 4 is a schematic view of the embodiment shown in FIG. 1.

FIG. 5 is a schematic view of the embodiment shown in FIG. 1.

FIG. 6 is a schematic view of the embodiment shown in FIG. 1.

FIG. 7A is a view of an embodiment showing an adjustment device.

FIG. 7B is a view of an embodiment showing a tool for use with an adjustment device.

FIG. 8 is a sectional view of the adjustment device of FIG. 7A.

FIG. 9A is a sectional view of the tool device of FIG. 7B.

FIG. 9B is a view of an embodiment depicting a use of the adjustment device of FIG. 7A.

FIG. 10A is a view of an embodiment depicting a use of the adjustment device of FIG. 7A and the tool of 7B.

FIG. 10B is a view of an embodiment depicting the adjustment device of FIG. 7A.

FIG. 11 is a view of an embodiment depicting a use of the adjustment device of FIG. 7A and the tool of 7B.

FIG. 12 is a schematic view of an adjustable length suture.

FIG. 13 is a view of an embodiment depicting a method of mitral valve repair using the adjustable length suture of FIG. 12.

FIG. 14 is another view of an embodiment depicting a method of mitral valve repair using the adjustable length suture of FIG. 12.

FIG. 15 is another view of an embodiment depicting a method of mitral valve repair using the adjustable length suture of FIG. 12.

### DETAILED DESCRIPTION

The present disclosure relates to methods and apparatuses for providing mitral valve chordal repair that permits the mitral valve leaflets to be attached to the papillary muscles or ventricular wall in a manner that is both minimally invasive and/or that permits chordal length adjustments.

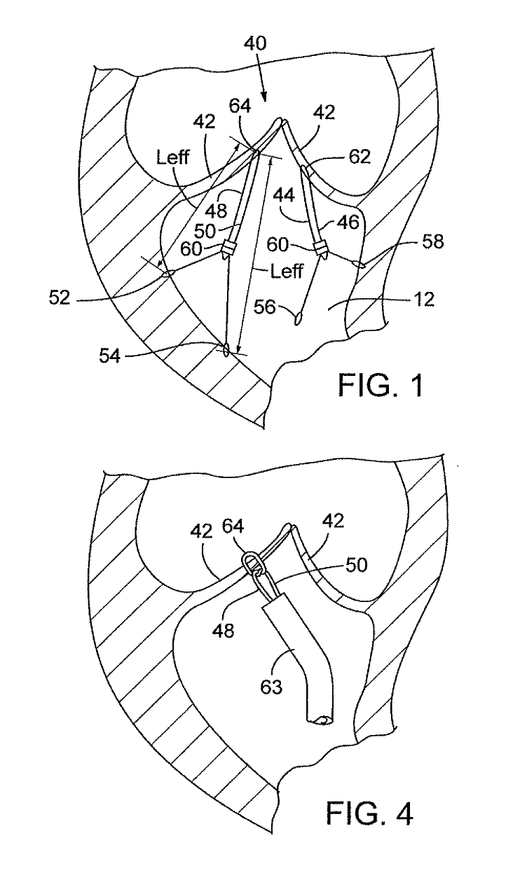

FIG. 1 discloses another novel method and apparatus for providing adjustable artificial chordae that can be implemented using a minimally invasive procedure. Two or more filaments (e.g., filaments 44, 46 and 48, 50) can be attached to a mitral valve leaflet at a common point or area (e.g., at fastening mechanisms 62 and 64) and then opposite ends of the filaments can be secured to the papillary muscle or ventricular wall at different locations (e.g., attachment points 52, 54 and 56, 58). An adjustment device 60 can be attached to each of the filaments to hold the filaments in close proximity to each other at the area where the adjustment device is in contact with the filaments. In this manner the effective length of each filament can be easily adjusted. The term “effective length” refers to the distance from the point of attachment of one end of a filament to the valve leaflet, to the point of attachment of the other end of the filament at or near a papillary muscle. FIG. 1 depicts the effective length L_{eff} of filaments 48 and 50. The effective length of filaments 48, 50 can be shortened by moving the adjustment device 60 in one direction (downward in FIG. 1), and lengthened by moving the adjustment device in the other direction (upwards in FIG. 1).

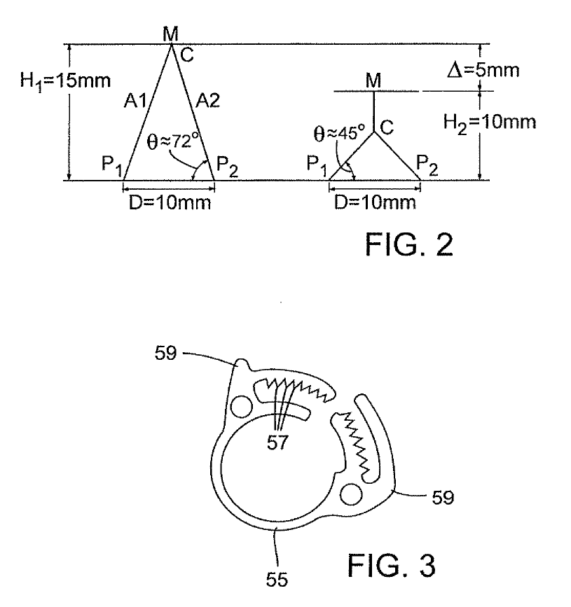

FIG. 2 illustrates the geometric relationship between an effective length H of two artificial chordae A1, A2 in relation to the position of an adjustment member C. The artificial chordae A1 and A2 are both attached at a common point (or area) M, which represents the point of attachment of the artificial chordae to a valve leaflet. The other ends of artificial chordae A1 and A2 are attached to different locations at or near the papillary muscle, P1, P2. In this example, locations P1 and P2 are at the same height inside the heart. As shown in FIG. 1, the lower ends of the artificial chords can be at different heights inside the left ventricle.

In the example shown in FIG. 2, when adjustment member C is positioned at the common point (or area) of attachment to a valve leaflet, the effective length H₁ of the artificial chordae A1, A2 is 15 mm. The angle θ defined by the line formed by points P1 and P2, and the line formed by P1 and the common point (or area) of attachment M is 72 degrees. If the adjustment member C is moved (or manipulated) along the artificial chordae A1, A2 so that adjustment member C is a lower position, then both the effective length H and the angle θ are changed. Specifically, effective length H₂ can be reduced, for example, to 10 mm and angle θ can be reduced, for example, to 45 degrees. By changing the location of adjustment member C relative to the artificial chordae in this manner, the effective length of the artificial chordae can be increased or decreased.

Referring again to FIG. 1, a mitral valve 40 with valve leaflets 42 is depicted. Filaments 44, 46, 48, 50 are attached within the left ventricle 12. To attach the filaments a catheter delivery system is provided with access to the left ventricle. This access is desirably gained through an incision in the apex of the heart in a transapical procedure. In such a procedure, an introducer sheath can be used to enter the left ventricle through an incision in the chest wall and ventricular wall. Two concentric rings of purse-string sutures can be used around the incision in the left ventricular wall to maintain a good seal around the introducer sheath. For convenience and to show the catheter systems disclosed herein more clearly, the introducer sheath is omitted from the figures. However, it should be noted that an introducer sheath can be used in each embodiment disclosed herein. A deployment catheter that contains the fastening mechanisms and one or more filament (artificial chordae) can pass through the introducer sheath into the left ventricle.

The deployment catheter can pass through the sheath and a distal end of the catheter can be advanced to a mitral valve leaflet. The valve leaflet can be captured on the distal end of the catheter by a vacuum system or some other capturing mechanism via the catheter. The catheter may have a steering mechanism that is operable to selectively bend or adjust the curvature of the catheter. Such a steering mechanism can assist in accessing the valve leaflets, as well as to help maneuver the catheter to the other areas of the heart or body discussed herein. Once the valve leaflet is captured, catheter deploys a fastening mechanism to be fastened to the valve leaflet.

Alternatively, the catheter delivery system can enter into the left ventricle through other known methods. For example, the mitral valve can be accessed percutaneously through a transfemoral procedure. In such a procedure, the left ventricle can be accessed through the left atrium using a deployment catheter Various procedures for gaining percutaneous access to the left atrium are known. For example, U.S. Patent Publication No. 2004/0181238, which is incorporated herein by reference, provides additional details for accessing the mitral valve via the femoral or jugular veins.

Two or more filaments can be attached to a common point (or area). In FIG. 1, filaments 44, 46 are shown attached to a common point (or area) on a valve leaflet 42 using fastening mechanism 62. Similarly, filaments 48, 50 are shown attached to a common point (or area) on a different valve leaflet 42 using fastening mechanism 64. For each common point (or area) of attachment, the attached filaments can be attached at the identical location or they can be attached in close proximity at substantially the same location. The attachment of multiple filaments at a common point (or area) can be achieved by a single fastening mechanism (as shown in FIG. 1) or by multiple fastening mechanisms.

The other end of filaments 44, 46 can be attached to the two different points 56, 58 on the papillary muscles or at their level on the ventricular wall, inferior to the mitral valve. Similarly, the other end of filaments 48, 50 can be attached to two different points 52, 54.

An adjustment member 60 can be attached to each of the two sets of filaments 44, 46 and 48, 50. The adjustment member can be a clamp, such as an adjustable, C-shaped clamp with interlocking teeth around a portion of the clamp. FIG. 3 depicts one embodiment of a C-shaped clamp 55. Clamp 55 has two arms 59, each formed with interlocking teeth 57. Interlocking teeth 57 are configured to lock the clamp in one or more positions of varying circumference when pressure is applied to the two arms 59 of the clamp pushing the two arms 59 together. As an adjustment member, the clamp can be tightened about the filaments so that the filaments are substantially fixed relative to one another. The inner surface of such a clamp desirably has grooves to increase the friction and decrease the slippage between the adjustment member and filaments 44, 46, 48, 50. Alternatively, a tension adjustment block could be used.

By moving the adjustment member up or down, the effective length of the artificial chordae can be varied. For example, by lowering the adjustment member along the filaments, the angles between the filaments and the plane of the two different points of attachment (e.g., 56, 58) is decreased and the overall effective length of the artificial chordae is decreased.

FIG. 4 depicts a catheter 63 that permits delivery of filaments 48, 50 to valve leaflet 42. In this embodiment, filaments 48, 50 comprise a single strand of filament that extends through a fastening mechanism 64. In this and in other embodiments, filaments 48, 50 can be a single strand or separate pieces of filament that are secured to the leaflet in close proximity to each other using separate fastening mechanisms. The specific method of attachment of filaments 48, 50 to the valve leaflet is not shown in FIG. 4, however, fastening mechanism 64 can be any known method, such as a clipping, stapling, barbed anchor, or other type of device that can pierce or can otherwise be secured to the leaflet. Alternatively, fastening mechanism 64 can be a tying or suture type fastener. The fastening mechanisms are desirably made of a material that is visible on fluoroscopy or other imaging technology to aid in placement of the fastener during the procedure.

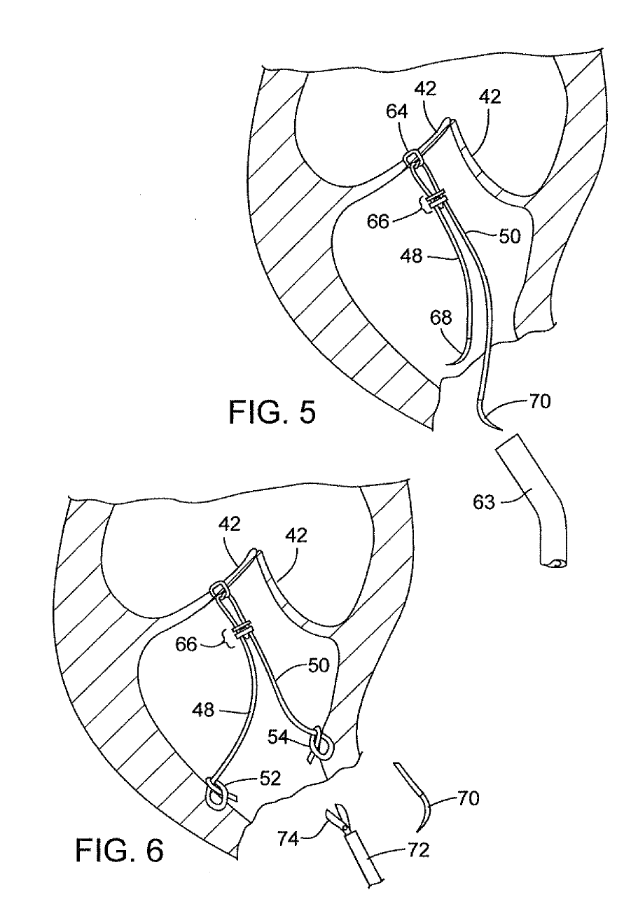

FIG. 5 shows filaments 48, 50 after their attachment to fastening mechanism 64 on the valve leaflet 42 and with the catheter delivery system pulled back to expose the free ends of filaments 48, 50. The free ends of filaments 48, 50 can optionally have a needle attachment 68, 70 to facilitate their attachment to the papillary muscle or ventricular wall. Adjustment member 66 depicted in FIG. 5 is a tension adjustment block, which is discussed in more detail below.

Referring to FIG. 6, after the free ends of filaments 48, 50 are attached (using the needle attachments 68, 70) at points 52, 54, a removal catheter 72 with a cutting device 74 can be used to remove loose, excess material of the free ends and the needle attachments (e.g., needle attachment 70). Although FIG. 6 depicts a suturing attachment method, the free ends of the filament alternatively could be attached by other means (such as those discussed above) to the papillary muscles or ventricular wall.

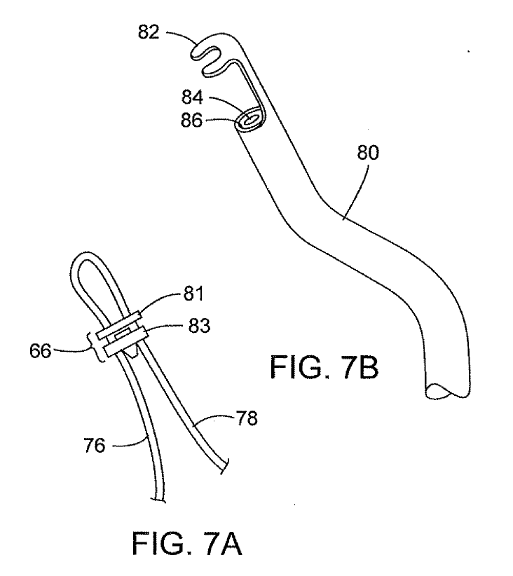

FIGS. 7-11 depict tension adjustment block 66 and an adjustment catheter 80. Referring to FIGS. 7A and 7B, tension adjustment block 66 is shown attached to two filaments 76, 78. Tension adjustment block 66 comprises a tapered, plastic pin 81 that fits into a tapered, plastic snap ring 83. When pin 81 and ring 83 are locked together, the tension adjustment block is prevented from moving relative to the filaments.

As best shown in FIG. 7B, adjustment catheter 80 in the illustrated configuration has a fork member. 82, an unlocking push member 84 that extends through fork member 82, and a locking push member 86 that extends through push member 84. Fork member 82 is configured so that it can move the tension adjustment block 66 relative to the filaments to which it is connected. In particular, fork member 82 can engage tension adjustment block 66 when it is positioned along the filaments (but not yet in a locked position) such that by moving the adjustment catheter in one direction along the length of the filaments the tension adjustment block is also moved. By moving tension adjustment block 66 in this manner, the effective length of the filaments can be changed.

Push members 84, 86 are movable longitudinally relative to each other and the fork member 82 to effect locking and unlocking of the adjustment block 66, as further described below The unlocking push member 84 unlocks the tension adjustment block from the locked position and the locking push member 86 locks the tension adjustment block from the unlocked position.

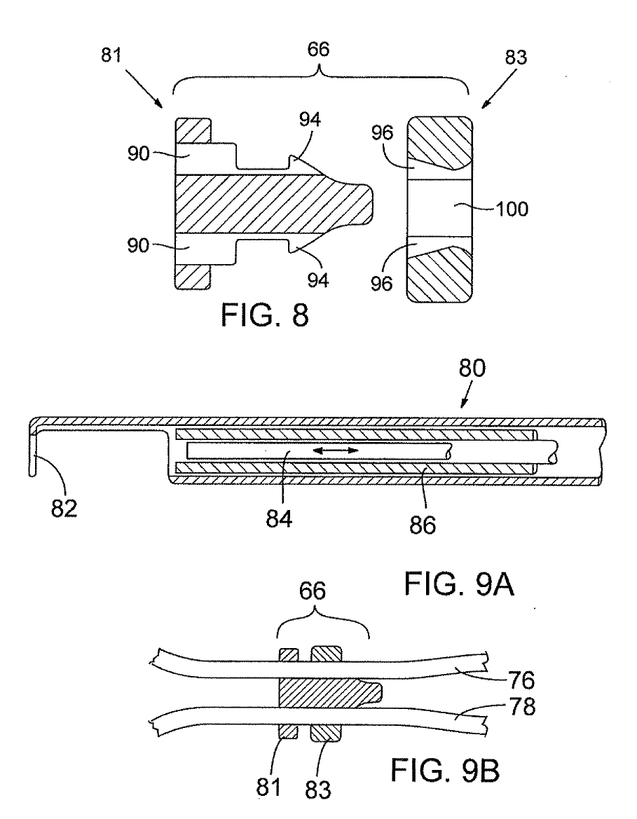

FIG. 8 depicts tension adjustment member, according to one embodiment, in more detail. Pin 81 comprises pin filament slots, or holes, 90 (which accept the filaments) and locking members, or flanges, 94 (which extend outward to secure the pin to the ring in a locked position). Ring 83 comprises ring filament slots, or holes, 96 (which accepts the filaments) and pin receiving hole 100 (which receives the pin to secure the pin to the ring in a locked position). The locking members 94 are deformable to allow the pin member to be inserted throughout ring member and form a snap-fit connection sufficient to hold the ring member on the pin member.

FIGS. 9-11 depict the relationship between tension adjustment block 66 and adjustment catheter 80, according to one embodiment, and their functions relative to one another. As discussed above, adjustment catheter 80 comprises fork member 82, unlocking push member 84, and locking push member 86. FIG. 9A shows the adjustment catheter 80 in more detail. Both unlocking push member 84 and locking push member 86 are movable within adjustment catheter 80 along the longitudinal direction identified by the arrow shown in FIG. 9A. Unlocking push member 84 is desirably a solid member that is sized to fit within the locking push member 86, which is desirably cylindrical with a longitudinally extending hollow section or lumen for receiving member 84.

FIG. 9B shows tension adjustment member 66 with the pin 81 and ring 83 locked together. In the locked position, filaments 76, 78 pass inside ring 83 and around pin 81 (through the ring filament holes and pin filament holes) and are captured between these two components. Filaments 76, 78 are held in place relative to each other and the pin and ring are held in place relative to the filaments by the friction created at the surface interfaces. In the locked position, tension adjustment block 66 acts to maintain the distance between the valve leaflets and the papillary muscles or ventricular wall.

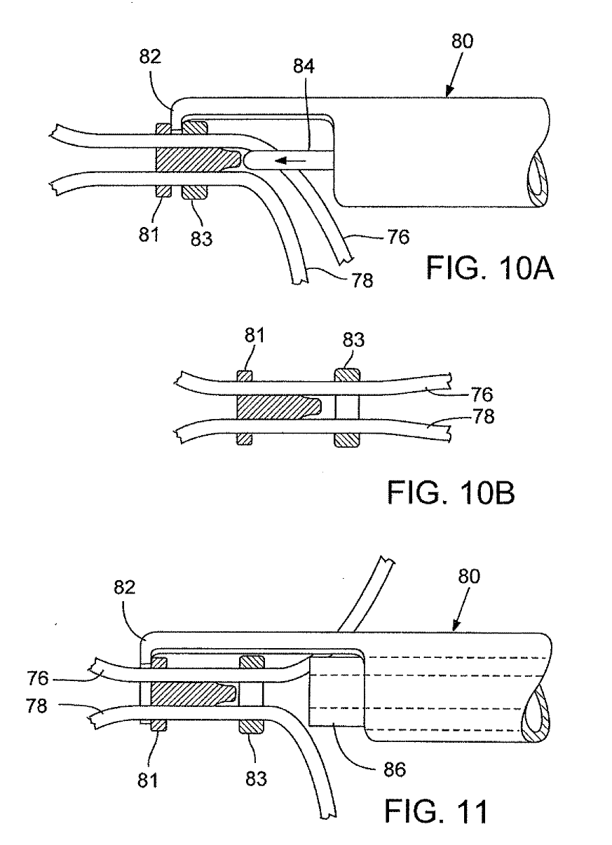

Referring to FIGS. 10A and 10B, to unlock tension adjustment member (i.e., separate pin 81 and ring 83), fork member 82 is inserted between pin 81 and ring 83 and unlocking push member 84 is extended from adjustment catheter 80 to push pin 81 and ring 83 apart. Fork member 82 holds the ring 83 in place, while unlocking push member 84 applies longitudinal pressure against the tip of pin 81, forcing it out of the ring 83. Unlocking push member 84 is desirably sized so that it can fit at least partially through the pin receiving hole 100 to assist in unlocking the pin and ring from one another. Once pin 81 and ring 83 are separated, tension adjustment member 66 can be moved relative to filaments 76, 78 in order to adjust the tension in the filaments.

Referring to FIG. 11, the manner in which adjustment catheter 80 can be used to secure pin 81 and ring 83 together is shown. Fork member 82 is placed at the far (distal) end of pin 81 and locking push member 86 is extended from the adjustment catheter 80. Locking push member is configured with a cylindrical surface that is sized to mate with the area of the ring that surrounds the pin receiving hole 100. While fork member holds pin 81 in place, locking push member forces ring 83 onto pin 81 and locks the pin 81 and ring 83 together. Once the tension adjustment member is locked, the frictional engagement of the adjustment member with the filaments maintains the position of the adjustment member relative to filaments 76, 78.

The three point connection system described above permits a physician to perform fine adjustments of length of artificial chordae, thereby allowing more accurate adjustments. The clamp or tension adjustment block allow for both lengthening and shortening of the artificial chordae. Also, since the length of the chordae can be adjusted relatively easily after they are connected to the valve leaflets and papillary muscles (or ventricular wall), the initial length selection for the filaments does not have to be as accurate.

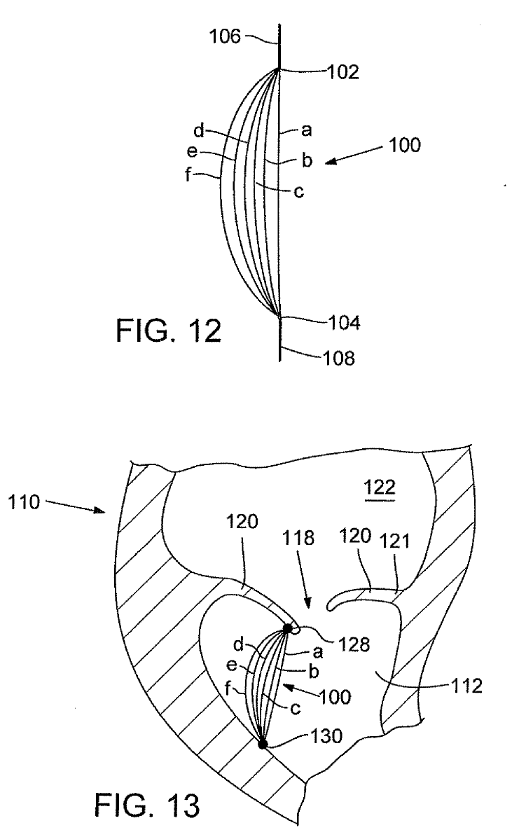

FIGS. 12-15 disclose another novel method and apparatus for providing adjustable artificial chordae that can be implemented using a minimally invasive procedure. FIG. 12 illustrates an adjustable length suture 100. Adjustable length suture 100 comprises multiple filament strands a, b, c, d, e, and f. Each individual strand can be composed of any suitable filament material, such as GORE-TEX® Sutures.

Each of strands a-f is desirably of a different length. As schematically illustrated in FIG. 12, strand a is the shortest strand, followed by strand b, then strand c, then strand d, then strand e, and then strand f, which is the longest strand of adjustable length suture 100. Each strand a-f is attached to common points (or areas) 102, 104 at opposite ends of suture 100.

Adjustable length suture 100 can have a filament portion that extends beyond common points (or areas) 102, 104. For example, FIG. 12 illustrates filaments 106, 108 extending beyond the common point. Filaments 106, 108 can be a single filament strand or they can be multiple strands.

As shown in FIGS. 13-15, adjustable length suture 100 can be used as an adjustable artificial chordae to correct mitral valve deficiencies in a heart. FIG. 13 depicts a schematic portion of a human heart 110. Heart 110 includes left ventricle 112. The mitral valve 118 includes valve leaflets 120, the mitral valve annulus 121, the papillary muscles (not shown), and the chordae tendineae (not shown). The chordae connect the valve leaflets to the papillary muscle in the left ventricle to prevent them from prolapsing into the left atrium 122.

As shown in FIG. 13, one end of the adjustable length suture 100 can be attached to a valve leaflet 120 and the other end adjustable length suture 100 can be attached at or near a papillary muscle. Adjustable length suture 100 can be attached to valve leaflet 120 by anchor mechanism 128 and at or near the papillary muscle by anchor mechanism 130. Anchor mechanisms 128, 130 can be any known attachment device, including those devices discussed above with regard to other embodiments. In addition, the left ventricle can be accessed to attach adjustable length suture 100 by any known method, including the transapical and transfemoral methods discussed above with respect to other embodiments.

Once attached via anchor mechanisms 128, 130, the effective length of the adjustable length suture 100 will be determined by the shortest length filament strand of adjustable length suture 100. In the example shown in FIG. 13, filament strand a, which is the shortest strand, limits the effective length of the adjustable length suture. Because the other filament strands b-f are longer than filament strand a, they will not be taut when adjustable length suture is attached to the valve leaflet 120 and papillary muscle via anchor mechanisms 128, 130.

Adjustable length suture 100 can be desirably attached so that the shortest length filament strand will be shorter than the desired effective length of the artificial chordae. Because the effective length of adjustable length suture 100 can be easily and conveniently lengthened in the manner discussed below, it is more desirable that the shortest length filament strand of adjustable length suture 100 be too short when first attached to the valve leaflets and papillary muscle, rather than too long.

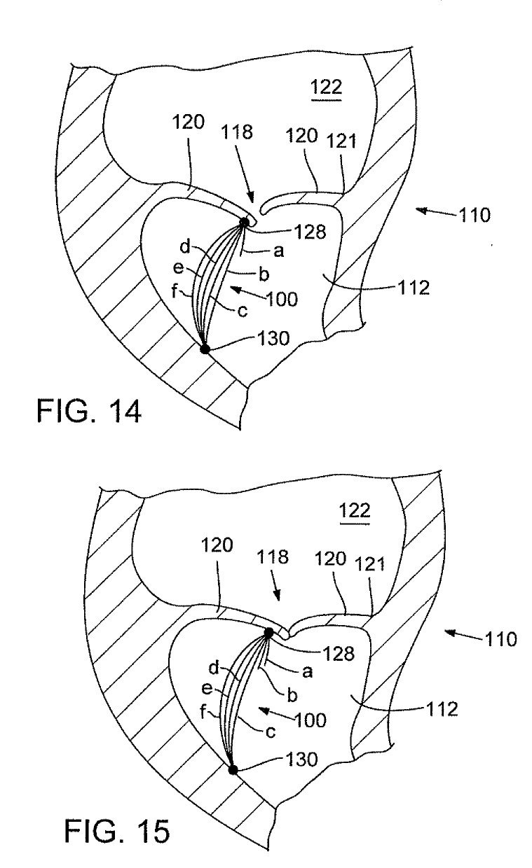

As shown in FIG. 13, anchor mechanism 128 is attached to valve leaflet 120. However, the effective length of adjustable length suture 100 in this example is shorter than the ultimate desired effective length and the valve leaflet is pulled into the left ventricle in an open position. To increase the effective length of adjustable length suture 100 a physician can cut the shortest filament strand. As shown in FIG. 14, strand a has been cut and strand b is now the shortest filament strand. Thus, the effective length of the adjustable length suture 100 is lengthened from the length of the shortest strand (strand a) to the length of the next shortest strand (strand b). As a result of lengthening the effective length of adjustable length suture 100, valve leaflet 120 moves closer to its natural position.

The heart can then be observed and, if the physician observes that the effective length of the adjustable length suture is still too short, the above cutting step can be performed again. As shown in FIG. 15, filament strand b can also be cut. The shortest filament strand is now filament strand c. Therefore, the length of filament strand c (the shortest, uncut filament strand) now determines the effective length of adjustable length suture 100. As filament strand c was longer than filament strand b, the effective length of adjustable length suture 100 increases and valve leaflet moves further towards its natural position. As shown in FIG. 15, after cutting filament strand b, valve leaflet 120 moves into the desired closed position and, in this example, it would not be necessary to cut any further strands.

The above steps can be repeatedly performed until the desired effective length of adjustable length suture 100 is obtained, or until the adjustable length suture has only one strand left and, therefore, is no longer adjustable by cutting additional strands. In addition, more than one adjustable length suture 100 can be used. The additional adjustable length sutures 100 can be attached to the same valve leaflet 120 or to other valve leaflets.

Filament strands a-f are desirably marked or otherwise identifiable so that the operating physician can ensure that the correct filament strand is being cut. For example, filament strands can be marked in a manner that is visible with fluoroscopy or other imaging methods.

The adjustable length suture described above comprises six different filament strands. It is desirable that the adjustable length suture has at least four strands; however, the adjustable length suture can be formed with other numbers of filament strands. As long as the adjustable length suture has at least two filament strands, the length of the suture can be adjusted in the manner described above. The maximum number of strands is limited only by the practicality of attaching the device and accurately identifying the individual strands during the adjustment procedure.

In addition, the change in length from one filament strand to another can vary as desired. It is preferable that an adjustable length suture can have a total change in length (i.e., the difference in length between the shortest filament strand and the longest filament strand) of about 3-5 mm. For example, if an adjustable length suture has six filament strands (as shown in the illustrative embodiment) and each filament strand varied in length from the next one by 0.7 mm, then the adjustable length suture would be adjustable by cutting one or more strands up to a total length of 3.5 mm. It is desirable that the adjustable length suture be capable of changing the length of an artificial chord in an amount of approximately 13-22 mm. That is, it is desirable that the distance between the longest strand and the shortest strand is approximately 13-22 mm.

It may be desirable to have a variety of adjustment length sutures available with different numbers of filament strands, different length variations between strands, and/or different variations in total effective lengths so that a physician can select the adjustment length suture that is best suited to a particular patient's anatomy and/or the type of procedure that is to be performed.

The attachment of the filaments to the valve leaflets, as well as the attachment of the filaments to the papillary muscle area discussed herein can be achieved by using one or more tools that are inserted into the body via an introducer sheath. In addition, the use of the common term catheter throughout this specification does not preclude the use of multiple, different catheter tools or devices to achieve the various different acts discussed herein.

It should be noted that each of the apparatuses and methods disclosed herein, to the extent that they are not inconsistent with one another, can be combined and utilized together. For example, adjustable length suture 100 (shown in FIG. 12) can be combined with adjustment member 66 (shown in FIG. 6) resulting in an artificial chordae whose effective length can be adjusted by cutting filament strands of the adjustable length suture 100 as well as by adjusting the position of the adjustment member 66.

The filaments described in this disclosure can be any type of material appropriate for artificial chordae, such as GORE-TEX® Sutures, which are a microporous, nonabsorbable monofilament made of expanded polytetrafluoroethylene (ePTFE). Although each adjustment member is only shown attached to two filaments, it would be possible and may be desirable to attach the adjustment to three or more filaments to achieve similar benefits.

The methods discussed above depict both transapical and transfemoral approaches for placement of artificial chordae. It should be understood, however, that the techniques described above can be generally applied to methods other than those discussed above, so long as the approach results in access to the left ventricle. For example, the techniques discussed above are applicable if the left ventricle is accessed via the femoral artery and the aorta.

Desirably, in each of the above-described procedures a physician can observe the beating heart of the patient during the procedure to determine whether the length or position of the artificial chords (e.g., filaments or sutures) should be adjusted. Such observation of the heart can be achieved by any known imaging technology.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of performing mitral valve chordal repair on a patient, the method comprising:
inserting a catheter into a left ventricle of the patient, the catheter containing at least a first and second filament;
attaching a first end of the first filament to a mitral valve leaflet at a valve attachment site;
attaching a first end of the second filament to the mitral valve leaflet at or near the same location as the attachment of the first end of the first filament to the mitral valve leaflet;
attaching the second end of the first filament to a first attachment site at either a papillary muscle or along the ventricular wall;
attaching a second end of the second filament to a second attachment site at either a papillary muscle or the ventricular wall, the first and second attachment sites being different from one another; and
attaching an adjustment device to both the first and second filaments, the adjustment device being configured to hold portions of the first and second filaments in close proximity to each other at the area where the adjustment device contacts the first and second filaments, the adjustment device being movable along a length of the first and second filaments to adjust the tension in the filaments.

2. The method of claim 1, further comprising:
moving the adjustment device along a length of the first and second filaments such that the movement of the adjustment device causes the effective length of both the first and second filament to be either lengthened or shortened; and
locking the adjustment device in a locked state at a desired location along the first and second filaments, the locked state holding the first and second filaments securely together.

3. The method of claims 2, wherein the adjustment device can be unlocked and the act of moving the adjustment device can be performed more than once.

4. The method of claim 2, wherein the adjustment device is a clamp comprising interlocking teeth that are configured to lock the clamp in one or more positions of varying circumference.

5. The method of claim 2, wherein the adjustment device is a tension adjustment block, the tension adjustment block comprising a pin member and ring member, the pin member and ring member being configured to lock together in a locked state when the pin member is pushed into the ring member.

6. The method of claim 5, wherein the pin member and the ring member can be unlocked from the locked state so that the act of moving the adjustment member can be performed more than once.

7. The method of claim 6, wherein the pin member and ring member can be unlocked by inserting an adjustment catheter between at least a portion of the pin member and at least a portion of the ring member, and applying a separating force to one or both of the pin member and the ring member to separate the pin member from the ring member.

8. The method of claim 7, wherein the adjustment catheter comprises:
a fork member, the fork member having one or more prongs, the one or more prongs comprising the portion of the adjustment catheter that is inserted between the at least a portion of the pin member and the at least a portion of the ring member;
an inner push member, the inner push member being contained in a area of the adjustment catheter; and
an outer push member, the outer push member being of a greater diameter than the inner push member and the outer push member surrounding at least a portion of the inner push member,
wherein the inner push member is configured to engage the adjustment member to unlock the pin member from the ring member, and the outer push member is configured to engage the adjustment member to lock the pin member to the ring member.

9. A method of performing mitral valve chordal repair on a patient while the patient's heart is beating, the method comprising:
inserting a catheter into a left ventricle of the patient, the catheter containing at least one suture, the suture comprising at least two filament strands, the filament strands being attached to one another at a first common area at one end of the filament strands and at a second common area at the other end of the filament strands, the respective lengths of the filament strands between the first and second common areas being different from one another;
attaching a first end of the suture to a mitral valve leaflet;
attaching a second end of the suture to a papillary muscle or a ventricular wall; and
adjusting the effective length of the suture by cutting one or more of the filament strands, wherein at least one filament strand remains uncut.

10. The method of claim 9, wherein the suture comprises four or more filament strands.

11. The method of claim 9, wherein the difference between the length of the shortest filament strand and the longest filament strand is greater than 13 mm.

12. An apparatus for securing two or more artificial chordae filaments together such that an effective length of the filaments can be adjusted, the apparatus comprising:
a pin member, the pin member comprising an opening for receiving at least two filaments; and
a ring member, the ring member having an opening for receiving the at least two filaments,
the ring member being configured to receive at least a portion of the pin member,
wherein the apparatus can be placed in a locked state by inserting the pin member into the ring member, thereby capturing the filaments between the pin member and the ring member such that the pin member and the ring member are held at a fixed position relative to the filaments, and
wherein the apparatus can be placed in an unlocked state by separating the pin member and the ring member so the pin member and the ring member can be moved relative to the filaments.

13. A system for adjusting an effective length of artificial chordae within a patient, the system comprising:
(1) an adjustment member configured to secure two or more artificial chordae together, the adjustment member comprising:
a pin member, the pin member having a first end and a second end, the pin member comprising an opening for receiving at least two filaments; and
a ring member, the ring member having an opening for receiving the at least two filaments,
the ring member being configured to receive at least a portion of the first end of the pin member,
(2) a tool configured to lock and unlock the adjustment member, the tool comprising:
a fork member being positioned at the distal end of the tool and comprising one or more prongs;
an inner push member, the inner push member being contained within a lumen of the tool; and
an outer push member, the outer push member being of a greater diameter than the inner push member and the outer push member surrounding at least a portion of the inner push member,
wherein the tool is configured to manipulate the adjustment member between a locked and an unlocked position.

14. The system of claim 13, wherein when the adjustment member is in the unlocked position, the one or more prongs are configured to abut an outside surface of the first end of the pin member, and the outer push member is configured so that it can exert a force against the ring member in the direction of the fork member so that at least a portion of the first end of the pin member enters into at least a portion of the ring member, locking the pin and ring members together, and
wherein when the adjustment member is in the locked position, the one or more prongs are configured to be inserted between at least a portion of the pin member and at least a portion of the ring member, and the inner push member is configured so that it can exert a force against the first end of the pin member, unlocking the pin and ring members from one another.

15. An apparatus for securing a mitral valve leaflet to a papillary muscle, the apparatus comprising:
at least two filament strands, the filament strands being attached at a first common area
at one end of the filament strands and at a second common area at the other end of the filament strands,
wherein the respective lengths of the filament strands between the first and second common areas are different from one another.

16. The apparatus of claim 15, wherein the apparatus has four or more filament strands.

17. The apparatus of claim 15, wherein the difference between the length of the shortest filament strand and the longest filament strand is 13 mm or greater.

18. The apparatus of claim 15, wherein the at least two filament strands contain different markings that are visible under fluoroscopy so that the at least two filament strands can be distinguished from one another via fluoroscopy.
